(19) United States
(12) Plant Patent Application Publication
(10) Pub. No.: US 2003/0106120 P1
(43) Pub. Date: Jun. 5, 2003

(54) Hybrid tea rose plant named 'Intrepid Red'

(76) Inventor: James W. Coiner, La Verne, CA (US)

Correspondence Address: CHRISTIE, PARKER & HALE, LLP, 350 WEST COLORADO BOULEVARD, SUITE 500, PASADENA, CA, 91105, US

(21) Appl. No.: 10/015,928

(22) Filed: Dec. 5, 2001

(51) Int. Cl. A01H 5/00

(52) U.S. Cl. PLT/140

(57) ABSTRACT

A new and distinct Hybrid Tea Rose plant variety with dark red flowers.

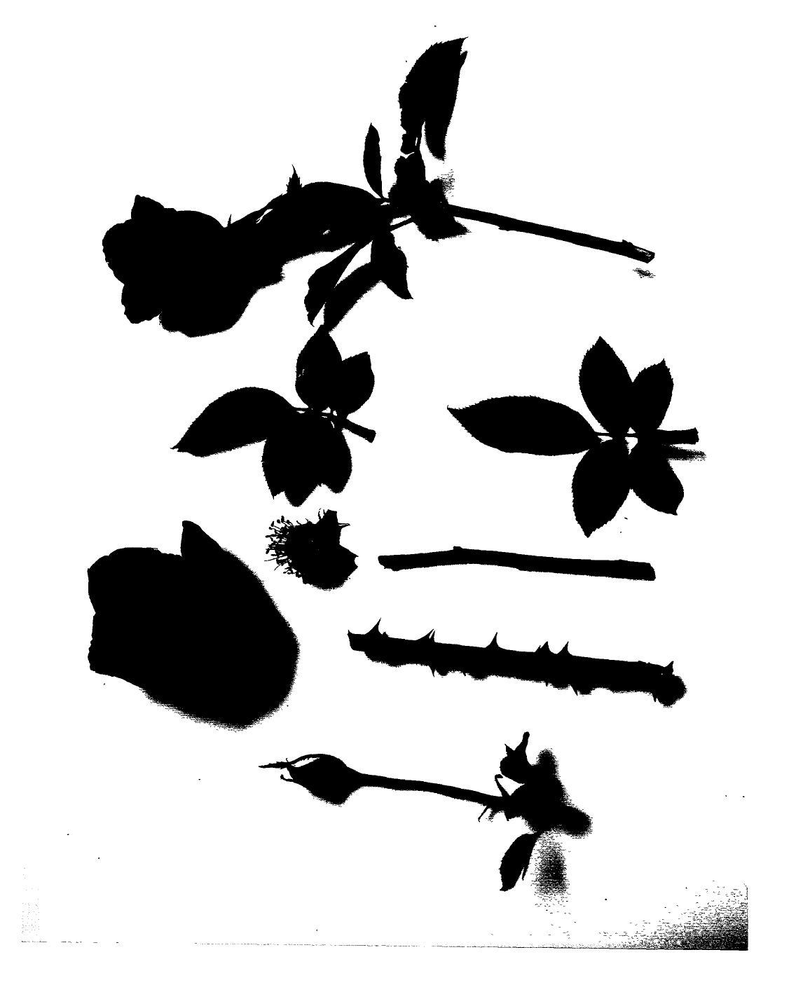

### BACKGROUND OF THE INVENTION

The present invention relates to a new and distinct variety of rose plant of the hybrid tea rose which was created by crossing as seed and pollen parents two (2) unknown rose varieties. The varietal denomination of the new variety is ‘INTREPID RED’.

### SUMMARY OF THE INVENTION

Among the novel characteristics possessed by this new variety which distinguishes it from other varieties of which I am aware, is its dark red flowers.

Asexual reproduction by propagation by budding of the new variety as performed in Wasco, Calif. shows that the foregoing and other distinguishing characteristics come true to form and are established and transmitted through succeeding propagations.

### BRIEF DESCRIPTION OF ILLUSTRATION

The accompanying illustration shows typical specimens of the flowers of the new variety depicted in color as nearly true as it is reasonably possible to make the same in a color illustration of this character.

### DESCRIPTION OF THE NEW VARIETY

The following is a detailed description of 2-year budded plants of the new variety, with color terminology in accordance with The Royal Horticultural Society Colour Chart (RHSCC). The terminology used in color description herein refers to plate numbers in the aforementioned color chart. Phenotypic expression may vary with light availability, environmental and cultural conditions.

The following observations are made of specimens grown outdoors in Wasco, Calif., during November.

### PLANT

Form: Compact

Growth: Medium vigor

Size: Height about 48 inches; width about 32 inches

Foliage:

Size.—Length about 2 3/16 inches; width about 1¼ inches.

Quantity.—About 5.

Color.—New foliage: Upper side near 136A; under side near 136B Mature foliage: Upper side near 137A; under side near 137C.

Shape.—Elliptic.

Texture.—Upper side: Smooth Under side: Matte.

Edge.—Serrated.

Serration.—Slight.

Petioles: Top surface color near 138B; Under side near 138B

Stipules: Medium length

Rachis: Few prickles

Variegation: None observed

### WOOD

New shoots:

Bark.—Smooth.

Wood.—Near 47B.

Old wood:

Bark.—Smooth.

Wood.—Near 140B.

Flowering stem: Length about 5 inches

Winter Hardiness: Completely winter hardy

Resistance to Disease: Average

Temperature range: Between 16° and 120° F.

Preferred growing conditions: Full sun

Growth habit: Compact

Leaves per stem: About 5

### FLOWER/INFLORESCENCE

Blooming Habit: Continuous

Bud.—Length about 1½ inches; width about 1½ inches Form: Classic rose Color: Before sepals divide color near 53A; when sepals first divide color near 53B; when petals begin to unfurl color near 53A.

Sepals: Pointed and serrated

Size.—Length in relation to bud, about 1¾ inches.

Color.—Topside near 156D; bottom near 138C.

Peduncle:

Length.—About 2 inches.

Aspect.—Strong, erect.

Color.—Near 141D.

Flower:

Size.—About 4¼ inches.

Borne.—Single — one per stem.

Form.—High centered.

Petalage: About 29

Petaloids in center.—Few.

Persistence.—Average.

Fragrance: Slight

Lasting Quality: About 3 weeks on plant; about 7 days after cutting

Color:

During first 2 days.—Inner petals at flower center topside near 60C; bottom near 63A; outer petals topside near 60C, bottom near 63A.

Base of petals (Point of attachment): Near 2B

General tonality from a distance: Near 63A

Color Change: As bloom ages to near 63A; after 3 or more days color near 63A

Petals:

Texture.—Soft.

Shape.—Longer than wide.

Arrangement.—Imbricated.

Appearance.—Velvety front; satiny back.

### REPRODUCTIVE ORGANS

Arrangement of stamens: Regularly arranged around

Filaments and Anthers: styles

Color: Filaments near 5B; Anthers near 6B

Styles: Medium length

Hips/Seed Vessels/Ovaries:

Shape.—Round.

Size.—About ¼ inch.

Color.—Near 141C.

Seeds: No seeds

I claim:

1. A new and distinct Hybrid Tea Rose plant of the variety substantially as shown and described.
